(19) United States
(12) Patent Application Publication
(10) Pub. No.: US 2003/0127332 A1
(43) Pub. Date: Jul. 10, 2003

(54) Method for producing multilayer paint coatings on electrically conductive substrates

(76) Inventors: Wolfgang Bremser, Munster (DE); Karl-Heinz Grosse-Brinkhaus, Nottuln (DE); Egon Wegner, Veitshochheim (DE)

Correspondence Address: BASF CORPORATION, ANNE GERRY SABOURIN, 26701 TELEGRAPH ROAD, SOUTHFIELD, MI, 48034-2442, US

(21) Appl. No.: 10/220,164

(22) PCT Filed: Feb. 17, 2001
(86) PCT No.: PCT/EP01/01769

(51) Int. Cl. C07K 1/26; G01L 1/20; C02F 1/469; G01F 1/64

(52) U.S. Cl. 204/502; 204/504

(57) ABSTRACT

The invention relates to a method for producing multilayer paint coatings according to which: (1) an electrophoretic paint that can be cathodically deposited is deposited on an electrically conductive substrate; (11) at least one coating material is applied to the resulting elecetrophonaic paint coat; (111) the electrophoretic paint coat and the at least one coat consisting of the coating material are subsequently hardened together. The electrophoretic paint contains an aqueous dispersion and can be produced by polymerizing at least one ethylenically unsaturated monomer in an aqueous solution of a protonized epoxy amine adduct, whereby the epoxy amine adduct can be obtained by reacting: (A) at least one glycidyl ether of a polyphenol which, in the statistical mean, contains at least one epoxy group in the molecule; (B) at least one polyglycidyl ether of a polyol which, in the statistical mean. contains more than one epoxy group in the molecule, and; (C) at least one compound which contains a primary amino group in the molecule, in order to form the epoxy amine adduct

A process for producing multicoat paint systems, in which

(I) a cathodically depositable electrocoat material is deposited on an electroconductive substrate,

(II) at least one coating material is applied to the resultant electrocoat film, and then

(III) the electrocoat film and the at least one film of the coating material are jointly cured, the electrocoat material comprising an aqueous dispersion preparable by polymerizing at least one ethylenically unsaturated monomer in an aqueous solution of a protonated epoxy-amine adduct, the epoxy-amine adduct being obtainable by reacting

(A) at least one glycidyl ether of a polyphenol, containing on average at least one epoxide group in the molecule,

(B) at least one polyglycidyl ether of a polyol, containing on average more than one epoxide group in the molecule, and (C) at least one compound containing a primary amino group in the molecule to give the epoxy-amine adduct.

### DESCRIPTION

The present invention relates to a novel wet-on-wet process for producing multicoat paint systems on electroconductive substrates.

Wet-on-wet processes for producing multicoat paint systems on electroconductive substrates, in which

(I) a cathodically depositable electrocoat material is deposited on an electroconductive substrate,

(II) at least one coating material is applied to the resultant electrocoat film, and then

(III) the electrocoat film and the at least one film of the coating material are jointly cured, are known.

For instance, Japanese patent application 1975-142501 (Japanese laid-open specification JP 52-065534 A2, Chemical Abstracts No. 87: 137427) describes a wet-on-wet process using an electrocoat material comprising an epoxy-amine adduct binder and a blocked polyisocyanate crosslinking agent and also an aqueous coating material comprising a neutralized polyester binder, a melamine resin crosslinking agent, and pigments.

American patent U.S. Pat. No. 4,375,498 A1 discloses a wet-on-wet process using an electrocoat material of the type described above and aqueous or conventional coating materials based on resins containing epoxide groups.

American patent U.S. Pat. No. 4,537,926 A1 describes a wet-on-wet process wherein unspecified cathodic electrocoat materials are overcoated with a coating material comprising special latex binders.

American patent U.S. Pat. No. 4,761,212 A1 discloses a wet-on-wet process using a cathodic electrocoat material which can be crosslinked with polyisocyanates. The electrocoat material itself, however, contains no compounds of that kind. The electrocoat film is overcoated with a two-component system comprising an isocyanate-reactive binder and polyisocyanates. Some of the polyisocyanates then diffuse into the electrocoat film and crosslink it.

European patent EP 0 529 335 A1 or German patent DE 41 25 459 A1 discloses a wet-on-wet process using a cathodic electrocoat material based on epoxy-amine adducts and blocked polyisocyanates and also an aqueous coating material based on a water-dilutable binder and a melamine resin comprising polyamide or polyacrylonitrile powder.

European patent EP 0 595 186 A1 or German patent application DE 42 35 778 A1 describes a wet-on-wet process using customary cathodic electrocoat materials and aqueous coating materials which crosslink on curing with the formation of urethane groups. In this case it is necessary, however, to employ specific pigment/binder ratios, and the baking temperature of the coating material must be higher than that of the electrocoat material.

A comparable wet-on-wet process, in which a powder coating material is used instead of the aqueous coating material, is known from European patent EP 0 646 420 A1. In this process as well the baking temperatures of the electrocoat material and of the powder coating material must be adapted precisely to one another.

Yet another wet-on-wet process, which uses a cathodic electrocoat material and an aqueous coating material, is known from European patent EP 0 639 660 A1.

European patent EP 0 817 648 A1 or German patent DE 195 12 017 C1 describes a wet-on-wet process in a first stage of which a cathodic electrocoat material and a color and/or effect coating material are applied and jointly baked before in a second stage a further color and/or effect coating material and a pigment-free coating material are applied and likewise jointly baked. In this process, the overall coat thickness of the two color and/or effect coatings must not be above or below a certain thickness, and the film thickness of the first color and/or effect coating must amount to from 20 to 50% of the total dry film thickness of the two color and/or effect coatings.

A disadvantage of all of these processes is that the cathodic electrocoat materials shrink on curing, as a result of which the roughness of the underlying electroconductive substrate surface is reproduced in the electrocoat. European patent EP 0 192 113 A2 therefore proposes a cathodic electrocoat material which exhibits only a low shrinkage on curing. This is achieved through the use of blocked polyisocyanates, blocked with low molecular mass blocking agents such as ethanol, for example. The European patent does not disclose a wet-on-wet process.

However, this concept is taken up again in German patent DE 41 26 476 A1. It describes a wet-on-wet process using a cathodic electrocoat material which, owing to the use of polyepoxide or polyisocyanate crosslinking agents blocked with low molecular weight blocking agents exhibits only a low shrinkage on curing. Disadvantageous for this known wet-on-wet process is the fact that on the one hand it is restricted to the use of specific crosslinking agents and on the other hand it is restricted to the use of specific aqueous coating materials, and therefore cannot be broadly employed.

International patent application WO 98/07794 discloses a cathodic electrocoat material comprising a dispersion preparable by

(1) polymerizing an ethylenically unsaturated monomer or a mixture of ethylenically unsaturated monomers in

(2) an aqueous solution of an at least partly protonated epoxy-amine adduct,

(3) the epoxy-amine adduct being obtainable by reacting

(A) at least one glycidyl ether of a polyphenol, containing on average at least one epoxide group in the molecule,

(B) at least one polyglycidyl ether of a polyol, containing on average more than 1.0 epoxide group in the molecule, and

(C) at least one compound containing a primary amino group in the molecule,

to give the epoxy-amine adduct, components (A) and (B) being used in an equivalents ratio of from 1.0:0.5 to 1.0:8.0, and using from 0.3 to 0.7 mol of component (C) per equivalent of epoxide groups of (A) and (B).

The electrocoats produced from the known electrocoat material feature an improved profile of technomechanical properties in terms of the adhesion, hardness, flexibility, and stonechip resistance, and also the corrosion protection and the edge protection.

A wet-on-wet process of the type described at the outset is not described in the international patent application. No details are given of the smoothness of the surface of the baked electrocoat. Moreover, the international patent application does not indicate which coating materials might be applied to the electrocoat and what effect the electrocoat has on the smoothness of the coatings.

It is an object of the present invention to find a novel wet-on-wet process, of the type described at the outset, which no longer has the disadvantages of the prior art but which instead provides smooth multicoat paint systems on electrically conductive substrates even at low film thicknesses, substantially or completely independently of the structure of the crosslinking agents of the cathodic electrocoat materials, especially of the blocked polyisocyanates. As far as the coating materials which can be processed by this process are concerned, moreover, the novel wet-on-wet process ought to be capable of very broad application. The invention accordingly provides the novel wet-on-wet process for producing multicoat paint systems on electroconductive substrates, in which

(I) a cathodically depositable electrocoat material is deposited on the electroconductive substrate,

(II) at least one coating material curable thermally or both thermally and with actinic radiation is applied to the resultant electrocoat film, and then

(III) the electrocoat film and the film of the coating material, or the two said films and at least one further, overlying film of a coating material, are jointly cured,

the cathodically depositable electrocoat material comprising an aqueous dispersion preparable by

(1) polymerizing an ethylenically unsaturated monomer or a mixture of ethylenically unsaturated monomers in

(2) an aqueous solution of an at least partly protonated epoxy-amine adduct,

(3) the epoxy-amine adduct being obtainable by reacting

(A) at least one glycidyl ether of a polyphenol, containing on average at least one epoxide group in the molecule,

(B) at least one polyglycidyl ether of a polyol, containing on average more than 1.0 epoxide group in the molecule, and

(C) at least one compound containing a primary amino group in the molecule,

to give the epoxy-amine adduct, components (A) and (B) being used in an equivalents ratio of from 1.0:0.5 to 1.0:8.0, and using from 0.3 to 0.7 mol of component (C) per equivalent of epoxide groups of (A) and (B).

The novel wet-on-wet process for producing multicoat paint systems on electrically conductive substrates is referred to below as “process of the invention”.

Further subject matter of the invention will emerge upon reading the description.

In the light of the prior art it was surprising and unforeseeable for the skilled worker that the object on which the present invention is based could be achieved by means of the process of the invention. A particular surprise was that the advantages obtained by the process of the invention are not tied to the use of specific crosslinking agents. Even more of a surprise was the extremely broad applicability of the process of the invention, particularly as regards the coating materials which can be used in said process.

In a first step of the process of the invention, a cathodically depositable electrocoat material is deposited on an electrically conductive substrate.

The substrates may be composed of any materials whose electroconductivity allows such a deposition. Examples of suitable substances are metals, electroconductive plastics or electroconductive ceramic materials, but especially metals. The substrates may be employed in any of a very wide variety of technical fields. In particular, they constitute bodies of motor vehicles or parts of motor vehicle bodies, furniture made of metal, or industrial components such as coils, containers or packaging, nuts, bolts, parts of tools, hubcaps, radiators or sheet-metal sections for interior and exterior use on buildings.

The apparatus for cathodic electrodeposition coating, and the conditions employed therefor, are customary and are described in detail, for example, in the patents cited at the outset.

Similarly, the cathodic electrocoat materials which may be employed in the process of the invention are customary and are likewise described in the patents cited at the outset. Further examples of suitable cathodic electrocoat materials are disclosed, for example, on page 3 lines 54 to 58 of European patent EP 0 595 186 A1 or in European patents EP 0 074 634 A1 and EP 0 505 445 A1.

The inventively essential constituent of the cathodic electrocoat material employed in the process of the invention is the aqueous dispersion.

It is obtainable by polymerizing an ethylenically unsaturated monomer or a mixture of ethylenically unsaturated monomers in an aqueous solution of an at least partly protonated epoxy-amine adduct, the epoxy-amine adduct being obtainable by reacting (A) a glycidyl ether from a polyphenol, containing on average at least one epoxide group in the molecule, or a mixture of such glycidyl ethers, (B) a polyglycidyl ether of a polyol, containing on average from 1.0 epoxide group in the molecule, or a mixture of such polyglycidyl ethers, and (C) a compound containing a primary amino group in the molecule, or a mixture of such compounds, to give the epoxy-amine adduct, components (A) and (B) being used in an equivalents ratio of from 1.0:0.5 to 1.0:8.0, and using from 0.3 to 0.7 mol of component (C) per equivalent of epoxide groups of (A) and (B).

One epoxy-amine adduct of the construction specified above is known per se from European patent application EP 0 505 445 B1.

Ethylenically unsaturated monomers are, for example, substances from the group consisting of “aliphatic and aromatic ethylene derivatives, alkyl acrylic esters, alkyl methacrylic esters, hydroxyalkyl acrylic esters, hydroxyalkyl methacrylic esters, halogenated forms of said monomers” or mixtures thereof, preferably diene-free compounds, especially styrene. Styrene is first inexpensively available and secondly can be used to prepare dispersions having outstanding properties. Examples of suitable acrylic esters of the general formula H₂C═CH—COOR are methyl, ethyl, n-butyl, and isobutyl acrylic esters. Examples of hydroxyalkyl acrylic esters are hydroxyethyl and hydroxypropyl acrylic esters. The preferred methacrylic esters of the general formula

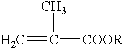

are methyl, butyl, hexyl, and octyl methacrylate. Examples of hydroxyalkyl methacrylates are hydroxyethyl and hydroxypropyl methacrylate.

The epoxy-amine adduct obtained from (A), (B), and (C) is preferably free from epoxide groups. Should it still contain epoxide groups, it is appropriate to react the remaining epoxide groups with compounds such as monophenols and amines, for example, especially secondary amines (examples of compounds suitable for reaction with remaining epoxide groups are listed in European patent EP 0 253 404 A1 on page 8 lines 28 to 37 and page 9 line 16 to page 10 line 15).

Preferred epoxy-amine adducts are obtained when components (A) and (B) are used in an equivalents ratio of from 1.0:1.0 to 1.0:2.0 and when component (C) is used in an amount such that there are from 0.4 to 0.6 mol of component (C) per equivalent of epoxide groups from (A) and (B).

The number-average molecular weight of the epoxy-amine adducts is preferably between 1 000 and 100 000, preferably between 3 000 and 15 000, daltons. Component (C) may be reacted in succession with (A) and (B) or—preferably—with a mixture of (A) and (B). The reaction of components (A) (B), and (C) may take place even at room temperature. In order to achieve economic conversion times it is appropriate to raise the reaction temperature, to 60 to 130° C., for example. The reaction of components (A), (B), and (C) is conducted where appropriate in an organic solvent such as ethylene glycol monobutyl ether or propylene glycol monobutyl ether, for example. The product is subsequently neutralized with an acid, such as acetic or lactic acid, for example, and converted into an aqueous dispersion or solution. The dispersion or solution thus obtained can then be processed further in accordance with conventional methods. It is also possible to mix the reaction product obtained from (A), (B), and (C), in solution in an organic solvent, with pigments and, where appropriate, fillers and to process it further by adding acid and, where appropriate, water to a pigment-containing dispersion. It is of course also possible to use mixtures of the epoxy-amine adducts.

As component (A) it is possible in principle to use any glycidyl ether of a polyphenol that contains on average at least one epoxide group in the molecule, or a mixture of such glycidyl ethers. As component (A) it is possible, for example, to use glycidyl ethers of the general structural formulae (I) and (II) that can be found on page 4 of European patent EP 0 253 404 A1. As component (A) it is preferred to use bisphenol A diglycidyl ethers, modified where appropriate with component (b) (see below), having an epoxide equivalent weight of from 180 to 3 000, preferably from 180 to 1 000. As component (A) it is particularly preferred to use mixtures of glycidyl ethers which are obtainable by preparing glycidyl ethers, in the presence of a catalyst of the reaction between phenolic hydroxyl groups and epoxide groups, from

(a) a diglycidyl ether of a polyphenol, preferably a diglycidyl ether of bisphenol A, having a number-average molecular weight of from 260 to 450, preferably from 370 to 380, or a mixture of such diglycidyl ethers,

(b) an unsubstituted or substituted monophenol, preferably an alkyl phenol having from 1 to 18, preferably from 4 to 12, carbon atoms in the alkyl radical, or a mixture of such monophenols, and

(c) a diphenol, preferably bisphenol A, and

(d) a catalyst of the reaction between aliphatic hydroxyl groups and epoxide groups,

the resultant glycidyl ethers having a number-average molecular weight of from 980 to 4 000, preferably from 980 to 2 000, and containing on average per molecule from 1.0 to 3.0, preferably from 1.2 to 1.6, epoxide groups and from 0.25 to 1.3, preferably from 0.4 to 0.9, phenyl ether groups originating from component (b). The component (A) used with particular preference is preferably prepared in organic solvents such as xylene, ethylene glycol monobutyl ether or propylene glycol monobutyl ether, for example. The reaction temperatures are appropriately 100-180° C. Catalysts (d) of the reaction between phenolic hydroxyl groups and epoxide groups are known to the skilled worker. Examples include the following: triphenylphosphine and the catalysts specified on page 9 in lines 6 to 9 of European patent EP 0 253 404 A1. Component (c) is intended to ensure that relatively high molecular mass glycidyl ethers are synthesized from component (a). This synthesis can be achieved by means of chain extension with a diphenol, preferably with bisphenol A. The synthesis may also take place, however, by reacting aliphatic hydroxyl groups present in component (a), and/or in the reaction product of (a) and (b), with epoxide groups. In order to be able to utilize this reaction in a targeted way to synthesize the desired glycidyl ethers, it is necessary to use catalysts (d) (e.g., tertiary amines) of the reaction between aliphatic hydroxyl groups and epoxide groups. Through the use of diphenol and a catalyst (d) it is possible to make use of both synthesis reactions—the chain extension by way of the diphenol and the addition reaction between aliphatic hydroxyl groups and epoxide groups. Reaction with component (b) is intended to modify the preferred glycidyl ethers and to lead to the formation of aliphatic hydroxyl groups, which are needed if it is intended that synthesis reactions should take place by way of addition reactions of aliphatic hydroxyl groups with epoxide groups. Through the statement of the number-average molecular weight of the particularly preferred component (A) to be prepared and the details concerning the epoxide groups present in component (A) and phenyl ether groups originating from component (b) it is readily possible for the skilled worker to calculate the amounts of (a), (b), and (c) to be used. If synthesis reactions proceeding by way of the reaction of aliphatic hydroxyl groups and epoxide groups are employed, it is necessary to terminate the synthesis reaction on reaching the epoxide equivalent weight which can be calculated by the skilled worker from the target number-average molecular weight and the target epoxide group content. Termination is appropriately effected by reducing temperature and diluting the reaction mixture.

As component (B) it is possible in principle to use any polyglycidyl ether of a polyol, containing on average more than 1.0 epoxide group in the molecule, or a mixture of such polyglycidyl ethers. As component (B) it is possible, for example, to use the polyglycidyl ethers described in European patent EP 0 253 404 A1 from line 42 of page 4 to line 13 of page 8. As component (B) it is preferred to use polyglycidyl ethers of polyetherpolyols, with particular preference diglycidyl ethers of polyetherdiols having number-average molecular weights of from 300 to 3 000, preferably from 400 to 1 200. Examples of particularly preferred polyglycidyl ethers include diglycidyl ethers of poly(ethylene glycol), poly(propylene glycol), poly(ethylene glycol-propylene glycol), and poly(1,4-butanediol), the number-average molecular weights of the diglycidyl ethers being between 300 to 3 000, preferably between 400 to 1 200.

As component (C) a compound is used which contains a primary amino group in the molecule, or a mixture of such compounds. Component (C) may contain only one primary amino group in the molecule. Besides the primary amino group, component (C) may contain further functional groups such as, for example, tertiary amino groups and hydroxyl groups. Component (C) is incorporated into the epoxy-amine adducts of the invention with the formation of tertiary amino groups. Here, one primary amino group reacts with two epoxide groups and so chain-extendingly links two molecules of components (A) and/or (B). Some of component (C) may also react with terminal epoxide groups to form secondary amino groups. As component (C) it is possible in principle to use any compound which contains one and only one primary amino group in the molecule. Examples include compounds of the general formula H₂N—CR₁R₂—R₃—O(CHR₄—CHR₅—O)ₙR₆: in this formula, R₁ and R₂ stand for hydrogen, alkyl or —CH—OH groups, R₃ stands for a linear or branched alkylene radical, particularly for an alkylene radical having from 1 to 3 carbon atoms, R₄ and R₅ stand for hydrogen or alkyl radicals having from 1 to 4 carbon atoms, R₆ stands for hydrogen or an alkyl, cycloalkyl or phenyl radical, preferably for an alkyl radical having from 1 to 6 carbon atoms, and n=0 to 5. Examples of suitable compounds of this type include ethanolamine, propanolamine, butanolamine, 2-amino-2-methylpropan-1-ol (H₂N—C(CH₃)₂—CH₂OH), 2-amino-2-ethylpropan-1-ol, and ethoxylated and/or propoxylated ethanol amine or propanol amine, such as, for example, 2-(2′-aminoethoxy)ethanol (H₂N—CH₂—CH₂—O—CH₂—CH₂—OH) and diethylene glycol mono-(3-aminopropyl)ether (H₂N—(CH₂)₃—O—CH₂—CH₂—O—CH₂—CH₂—OH). As component (C) it is also possible to use compounds which contain one primary and one tertiary amino group in the molecule. Examples include the following: N,N-dimethylamino-propylamine, N,N-diethylaminoethylamine, and the like. As component (C) it is also possible to use primary alkylamines such as hexylamine, for example. Unsubstituted or substituted aniline may also be used as component (C). As component (C) it is preferred to use hexylamine and N,N-dimethylaminopropylamine and also 2-(2′-aminoethoxy)ethanol.

In one preferred embodiment the dispersions are obtainable by using the ethylenically unsaturated monomer or the mixture of ethylenically unsaturated monomers and the at least partly protonated epoxy-amine adduct in a weight ratio of from 9.0:1.0 to 0.1:1.0, preferably from 5.0:1.0 to 1.0:1.0, with particular preference from 2.5:1.0 to 3.5:1.0.

In particular, styrene alone may be used as the ethylenically unsaturated monomer. Alternatively, as a mixture of ethylenically unsaturated monomers, a mixture of styrene and at least one further unsaturated monomer copolymerizable with styrene may be used. In the latter case, the mixture of ethylenically unsaturated monomers may contain advantageously at least 70% by weight, preferably at least 80% by weight, with particular preference at least 90% by weight, of styrene.

In a development of the invention, the ethylenically unsaturated monomer or the mixture of ethylenically unsaturated monomers may be polymerized free-radically using a water-insoluble initiator or a mixture of water-insoluble initiators. In this context it has proven very appropriate to use the water-insoluble initiator or the mixture of water-insoluble initiators in an amount of from 0.1 to 10.0% by weight, preferably from 0.5 to 5.0% by weight, with particular preference from 0.3 to 3.0% by weight, based on the amount of ethylenically unsaturated monomer used or on the amount of mixture of ethylenically unsaturated monomers used. Suitable initiators include all customary initiators for free-radical chain polymerization. Such initiators include in particular azo compounds, peroxides, hydroperoxides, and peresters, and also redox initiators. Particular preference is given to using azoisovaleronitrile as initiator.

In one preferred development of the invention, the aqueous dispersions are obtainable by introducing at least 50% by weight, preferably at least 75% by weight, with particular preference 100% by weight, of the total amount of initiator used, in the form of an initial charge, and adding the ethylenically unsaturated monomer or adding the mixture of ethylenically unsaturated monomers over the course of not more than 3 hours, preferably not more than 2 hours, with particular preference not more than one hour.

The amount of the dispersion in the cathodic electrocoat material for use in accordance with the invention may vary very widely and is guided by the requirements of the case in hand, such as result, for example, from the nature and amount of the other constituents. The cathodic electrocoat material preferably contains the dispersion in an amount of from 1.0 to 50% by weight, more preferably from 2.0 to 30% by weight, and in particular from 3.0 to 20% by weight, based in each case on the solids content of the cathodic electrocoat material.

The viscosity of the dispersion can be chosen as desired. At 23° C. and a dispersion solids content of from 50 to 60% by weight, for example, it is situated in the region above 5 000 mPa·s. In general, the viscosities may be up to 10 000 mPa·s. The polymerized ethylenically unsaturated monomers typically (but not mandatorily) have a mass-average molecular weight of more than 100 000. Individually, the polymer particles obtained advantageously have a size of up to 20 μm, preferably up to 10 μm. In order to bring about these conditions it is useful if the solids content of the epoxy-amine adduct solution or dispersion is in the range from 45 to 60% and the temperature during the polymerization of the monomers is in the range from 70° C. to 90° C. The particle size can be measured, for example, by means of light microscopy. A polymer particle size of this kind on the one hand makes it possible to achieve the necessary dispersion consistency, while on the other hand it has a positive effect on the properties of the electrocoats. A satisfactorily low electrocoat density is achieved when the weight ratios indicated above are observed. It is advantageous if the epoxy-amine adduct in the composition is similar or identical in structure to the binder of the deposition coating material.

The dispersions can be introduced into the electrocoat material by way of a paste resin, such as a pigment paste. They can be added to the pigment paste preferably before, during and/or after the milling process. However, they can also be used as the sole grinding resin. Introduction by way of the binder dispersion of the coating material is a further possibility.

Moreover, the cathodic electrocoat materials for use in accordance with the invention may comprise suitable additives, including pigments, such as are described in detail, for example, in international patent application WO 98/07794, page 13 lines 8 to 32, in the text book “Lackadditive” [Additives for Coatings] by Johan Bieleman, Wiley-VCH, Weinheim, New York, 1998, in Römpp Lexikon Lacke und Druckfarben, Georg Thieme Verlag, 1998, page 176: “Effect pigments”, pages 380 and 381: “Metal oxide-mica pigments” to “Metal pigments”, pages 180 and 181: “Iron blue pigments” to “Black iron oxide”, pages 451 to 453: “Pigments” to “Pigment volume concentration”, page 563: “Thioindigo pigments”, page 567: “Titanium dioxide pigments”, pages 250 ff.: “Fillers”, pages 623 and 624: “Aqueous coatings additives” or in European patent EP 0 693 540 A2, page 5 lines 6 to 10. These additives may also be present in the coating materials for use in accordance with the invention.

Following the deposition of the cathodic electrocoat material for use in accordance with the invention on the electroconductive substrate, the resultant electrocoat film is overcoated directly with at least one coating material curable thermally or both thermally and with actinic radiation. It may be rinsed with water beforehand. However, it may also be dried directly after deposition or after rinsing with water, without being crosslinked or cured. The electrocoat film is preferably dried before further films are applied.

In accordance with the invention more than one coating material can be applied to the electrocoat film. In this case it is preferred to employ coating materials which differ in composition and which fulfil different technological functions. The ultimate result of this are multicoat paint systems with three or more coats. In principle there is no upper limit on the number of coats. For economic reasons, however, the aim will be to restrict the number of coats to the necessary level, since each additional coat implies an additional economic and technical outlay. Generally speaking, however, four or five coats will be sufficient to meet even very complex technological requirements, such as are imposed, for example, in the OEM finishing of automobiles. Multicoat paint systems of this kind comprise, for example, an electrocoat, a surfacer coat or an antistonechip primer, a basecoat, a clearcoat, and, where appropriate, a highly scratch-resistant coating.

The coating material for use in accordance with the invention is pigmented. Examples of suitable pigmented coating materials are surfacers, antistonechip primers, basecoat materials, and solid-color topcoat materials.

Alternatively, the coating material for use in accordance with the invention may be unpigmented. Examples of suitable nonpigmented coating materials are clearcoat materials.

The coating material cures thermally (i.e., is thermosetting). In this case it may be self-crosslinking or externally crosslinking. In the context of the present invention, the term “self-crosslinking” refers to the capacity of a binder to undergo crosslinking reactions with itself. A prerequisite for this is that the binders already contain both kinds of complementary reactive functional groups which are necessary for crosslinking. Externally crosslinking coating materials, on the other hand, are those in which one kind of the complementary reactive functional groups is present in the binder and the other kind is present in a curing or crosslinking agent. For further details, refer to Römpp Lexikon Lacke und Druckfarben, Georg Thieme Verlag, Stuttgart, New York, 1998, “Curing”, pages 274 to 276, especially page 175, bottom.

Suitable binders are random, alternating and/or block, linear and/or branched and/or comb, addition (co)polymers of ethylenically unsaturated monomers, or polyaddition resins and/or polycondensation resins such as are described in Römpp Lexikon Lacke und Druckfarben, Georg Thieme Verlag, Stuttgart, New York, 1998, page 457: “Polyaddition” and “Polyaddition resins (polyadducts)”, pages 463 and 464: “Polycondensates”, “Polycondensation” and “Polycondensation resins”, and also pages 73 and 74: “Binders”.

Examples of suitable complementary reactive functional groups are compiled in the following overview. In the overview, the variable R stands for an acyclic or cyclic aliphatic radical, an aromatic radical and/or aromatic-aliphatic (araliphatic) radical; the variables R′ and R″ stand for identical or different aliphatic radicals or are linked with one another to form an aliphatic or heteroaliphatic ring.

### Overview

### Examples of Complementary Functional Groups Binder and Crosslinking Agent or Crosslinking Agent and Binder

| | |
|---|---|
| —SH | —C(O)—OH |
| —NH₂ | —C(O)—O—C(O)— |
| —OH | —NCO |
| —O—(CO)—NH—(CO)—NH₂ | —NH—C(O)—OR |
| —O—(CO)—NH₂ | —CH₂—OH |
| >NH | —CH₂—O-R |
| | —NH—CH₂—OH |
| | —NH—CH₂—O-R |
| | —NH(—CH₂—O-R)₂ |
| | —NH—C(O)—CH(—C(O)OR)₂ |
| | —NH—C(O)—CH(—C(O)OR) |
| | (—C(O)-R) |
| | —NH—C(O)—NR′R″ |
| | ═Si(OR)₂ |
| | 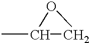 |
| —C(O)—OH | 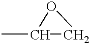 |

The selection of the respective complementary groups is guided on one hand by the consideration that they must not enter into any unwanted reactions during the storage and applications of the coating material and/or, where appropriate, must not disrupt or inhibit additional curing with actinic radiation, and on the other hand by the temperature range within which crosslinking is to take place.

In the case of the thermally curable coating materials it is preferred to employ crosslinking temperatures of from 100 to 200° C. Employed in the binders, therefore, are preferably thio, hydroxyl, methylol, methylol ether, N-methylol, N-alkoxymethylamino, imino, carbamate, allophanate and/or carboxyl groups, but especially carboxyl groups or hydroxyl groups, specifically hydroxyl groups, on the one hand, and employed in the crosslinking agents, therefore, are anhydride, carboxyl, epoxy, blocked isocyanate, urethane, methylol, methylol ether, N-methylol, N-alkoxymethylamino, siloxane, amino, hydroxyl and/or beta-hydroxyalkylamide groups, but especially blocked isocyanate groups or epoxy groups on the other. For the preparation of self-crosslinking binders it is preferred to use methylol, methylol ether, N-methylol or N-alkoxymethylamino groups.

Examples of suitable crosslinking agents are amino resins, as described, for example, in Römpp Lexikon Lacke und Druckfarben, Georg Thieme Verlag, 1998, page 29, “Amino resins”, in the text book “Lackadditive” by Johan Bieleman, Wiley-VCH, Weinheim, New York, 1998, pages 242 ff., in the book “Paints, Coatings and Solvents”, second, completely revised edition, edited by D. Stoye and W. Freitag, Wiley-VCH, Weinheim, New York, 1998, pages 80 ff., in patents U.S. Pat. No. 4,710,542 A1 or EP-B-0 245 700 A1, and in the article by B. Singh and coworkers, “Carbamyl-methylated Melamines, Novel Crosslinkers for the Coatings Industry”, in Advanced Organic Coatings Science and Technology Series, 1991, Volume 13, pages 193 to 207; carboxyl-containing compounds or resins, as described for example in patent DE 196 52 813 A1; compounds or resins containing epoxide groups, as described for example in patents EP 0 299 420 A1, DE 22 14 650 B1, DE 27 49 576 B1, U.S. Pat. No. 4,091,048 A1 or U.S. Pat. No. 3,781,379 A1; blocked polyisocyanates, as described for example in patents U.S. Pat. No. 4,444,954 A1, DE 196 17 086 A1, DE 196 31 269 A1, EP 0 004 571 A1 or EP 0 582 051 A1; and/or tris(alkoxycarbonylamino)triazines, as described in patents U.S. Pat. No. 4,939,213 A1, U.S. Pat. No. 5,084,541 A1, U.S. Pat. No. 5,288,865 A1 or EP 0 604 922 A1.

Further, the coating material is curable thermally and with actinic radiation, this being referred to by those in the art also as “dual cure”. In the context of the present invention, actinic radiation means electromagnetic radiation, such as visible light, UV radiation or X-rays, especially UV radiation, and corpuscular radiation such as electron beams.

The binders of the dual-cure coating materials, and also any reactive diluents present (cf. Römpp Lexikon Lacke und Druckfarben, Georg Thieme Verlag, Stuttgart, New York, 1998, page 491: “Reactive diluents”), contain groups which have at least one bond which can be activated with actinic radiation, which on exposure to actinic radiation becomes reactive and enters, with other activated bonds of its kind, into polymerization reactions and/or crosslinking reactions which proceed in accordance with free-radical and/or ionic mechanisms. Examples of suitable bonds are carbon-hydrogen single bonds or carbon-carbon, carbon-oxygen, carbon-nitrogen, carbon-phosphorus or carbon-silicon single bonds or double bonds. Of these, the carbon-carbon double bonds are particularly advantageous and are therefore used with very particular preference in accordance with the invention. For the sake of brevity they are referred to as “double bonds”.

Particularly suitable double bonds are present, for example, in (meth)acrylate, ethacrylate, crotonate, cinnamate, vinyl ether, vinyl ester, dicyclopentadienyl, norbornenyl, isoprenyl, isopropenyl, allyl or butenyl groups; dicyclopentadienyl ether, norbornenyl ether, isoprenyl ether, isopropenyl ether, allyl ether or butenyl ether groups; or dicyclopentadienyl ester, norbornenyl ester, isoprenyl ester, isopropenyl ester, allyl ester or butenyl ester groups. Of these, the acrylate groups offer very particular advantages, and so are used with particular preference.

The actinic radiation crosslinking may further be accelerated or initiated with suitable photoinitiators (cf. Römpp Lexikon Lacke und Druckfarben, Georg Thieme Verlag, Stuttgart, New York, 1998, pages 444 to 446: “Photoinitiators”).

The coating material may be a one-component (1K) system.

In the context of the present invention, a one-component (1K) system is a coating material which cures thermally or both thermally and with actinic radiation and in which the binder and the crosslinking agent are present alongside one another, i.e., in one component. A prerequisite for this is that the two constituents crosslink with one another only at relatively high temperatures and/or on exposure to actinic radiation.

The coating may further be a two-component (2K) or multicomponent (3K, 4K) system.

In the context of the present invention, this means a coating material in which in particular the binder and the crosslinking agent are present separately from one another in at least two components which are not combined until shortly before application. This form is chosen when binder and crosslinking agent react with one another even at room temperature.

Moreover, the coating material may be substantially free from water and/or organic solvent and may be pulverulent or liquid (100% system). In the context of the present invention, “substantially free” means that the amount of water and/or organic solvents is below 5.0%, preferably below 3.0%, more preferably below 2.0%, with particular preference below 1.0%, with very particular preference below 0.5%, by weight, and in particular below the gas-chromatographic detection limit.

Alternatively, the coating material may be a water-based coating material, especially an aqueous surfacer, aqueous basecoat material or aqueous powder coating dispersion (powder slurry).

Not least it may also comprise a conventional coating material, in other words a coating material based on organic solvents.

Examples of suitable surfacers or antistonechip primers to be used for the process of the invention are known from patents U.S. Pat. No. 4,537,926 A1, EP 0 529 335 A1, EP 0 595 186 A1, EP 0 639 660 A1, DE 44 38 504 A1, DE 43 37 961 A1, WO 89/10387, U.S. Pat. No. 4,450,200 A1, U.S. Pat. No. 4,614,683 A1 or WO 94/26827.

Examples of suitable color and/or effect basecoat materials, especially aqueous basecoat materials, to be used for the process of the invention are known from patents EP 0 089 497 A1, EP 0 256 540 A1, EP 0 260 447 A1, EP 0 297 576 A1, WO 96/12747, EP 0 523 610 A1, EP 0 228 003 A1, EP 0 397 806 A1, EP 0 574 417 A1, EP 0 531 510 A1, EP 0 581 211 A1, EP 0 708 788 A1, EP 0 593 454 A1, DE-A-43 28 092 A1, EP 0 299 148 A1, EP 0 394 737 A1, EP 0 590 484 A1, EP 0 234 362 A1, EP 0 234 361 A1, EP 0 543 817 A1, WO 95/14721, EP 0 521 928 A1, EP 0 522 420 A1, EP 0 522 419 A1, EP 0 649 865 A1, EP 0 536 712 A1, EP 0 596 460 A1, EP 0 596 461 A1, EP 0 584 818 A1, EP 0 669 356 A1, EP 0 634 431 A1, EP 0 678 536 A1, EP 0 354 261 A1, EP 0 424 705 A1, WO 97/49745, WO 97/49747, EP 0 401 565 A1 or EP 0 817 684, column 5 lines 31 to 45.

Examples of suitable one-component (1K), two-component (2K) or multicomponent (3K, 4K) clearcoat materials to be used for the process of the invention are known from patents DE 42 04 518 A1, U.S. Pat. No. 5,474,811 A1, U.S. Pat. No. 5,356,669 A1, U.S. Pat. No. 5,605,965 A1, WO 94/10211, WO 94/10212, WO 94/10213, EP 0 594 068 A1, EP 0 594 071 A1, EP 0 594 142 A1, EP 0 604 992 A1, WO 94/22969, EP 0 596 460 A1, EP 0 549 116 A2, EP 0 928 800 A1 or WO 92/22615.

Examples of suitable powder clearcoat materials to be used for the process of the invention are known, for example, from German patent DE-A-42 22 194 or the BASF Lacke+Farben AG product information “Pulverlacke”, 1990.

Examples of suitable powder slurry clearcoat materials to be used for the process of the invention are known, for example, from the US patent U.S. Pat. No. 4,268,542 and German patent applications DE 195 40 977 A1, DE 195 18 392 A1, DE 196 17 086 A1, DE-A-196 13 547, DE 196 52 813 A1, DE-A-198 14 471 A1.

Examples of suitable coating materials to be used for the process of the invention which produce highly scratch-resistant coatings are described in German patents DE 43 03 570 A1, DE 34 07 087 A1, DE 40 11 045 A1, DE 40 25 215 A1, DE 3828 098 A1, DE 40 20 316 A1 or DE 41 22 743 A1. Also suitable are organically modified ceramic materials, sold under the brand name ORMOCER®.

The coating material or materials can be applied by any customary methods, such as spraying, knifecoating, brushing, flowcoating, dipping, impregnating, trickling or rolling, for example. The substrate to be coated may itself be at rest, with the application equipment or unit being moved. Alternatively, the substrate to be coated, especially a coil, may be moved, with the application unit being at rest relative to the substrate or being moved appropriately.

The spray booth used for application may be operated, for example, with a circulation system, which may be temperature-controllable, and which is operated with a suitable absorption medium for the overspray, an example being the coating material itself.

Application is preferably carried out under illumination with visible light with a wavelength of more than 550 μm or in the absence of light, if the coating material is curable thermally and with actinic radiation. This prevents material alteration or damage to the aqueous basecoat material and the overspray.

In general, the coating materials are applied in a wet film thickness such that curing thereof results in coats having thicknesses which are advantageous and necessary for their functions. In the case of the surfacer coat or antistonechip primer coat this thickness is preferably from 10 to 150 μm, in the case of the basecoat it is preferably from 5 to 50, more preferably from 5 to 40, with particular preference from 5 to 30, and in particular from 10 to 25 μm, and in the case of the clearcoat it is preferably from 10 to 100, more preferably from 15 to 80, with particular preference from 20 to 75, and in particular from 25 to 70 μm. Highly scratch-resistant coatings generally have lower thicknesses, below 5.0 μm for example.

Following the application of the coating material or materials, the resulting coats are jointly cured.

Curing may take place after a certain rest time. This may have a duration of from 30 s to 2 h, preferably from 1 min to 1 h, and in particular from 1 min to 45 min. The rest time serves, for example, for leveling and devolatization of the films and for the evaporation of volatile constituents such as water and/or organic solvents. The rest time may be assisted and/or shortened through the use of elevated temperatures of up to 120° C. and/or by a reduced atmospheric humidity <10 g water/kg air, in particular <5 g/kg air, provided this does not entail any damage or alteration to the coating films, such as premature complete crosslinking.

The thermal cure has no special features in terms of its method but instead takes place in accordance with the customary and known methods such as heating in a forced air oven or irradiation using IR lamps. As with the actinic radiation cure described below, the thermal cure may also take place in stages. The thermal cure advantageously takes place at temperatures above 100° C. It is generally advisable here not to exceed temperatures of 200° C., preferably 190° C., and especially 185° C.

The actinic radiation cure is preferably conducted with UV radiation and/or electron beams. It employs a dose of preferably from 1 000 to 2 000, more preferably from 1 100 to 1 900, with particular preference from 1 200 to 1 800, with very particular preference from 1 300 to 1 700, and in particular from 1 400 to 1 600 mJ/cm². Where appropriate, this curing can be supplemented with actinic radiation from other radiation sources. In the case of electron beams it is preferred to operate under an inert gas atmosphere. This can be ensured, for example, by supplying carbon dioxide and/or nitrogen directly to the surface of the topmost coating film. In the case of curing with UV radiation as well it is possible to operate under inert gas, in order to prevent the formation of ozone.

The actinic radiation cure is carried out using the customary and known radiation sources and optical auxiliary measures. Examples of suitable radiation sources are flashlights from the company VISIT, high or low pressure mercury vapor lamps, which where appropriate had been doped with lead in order to open up a radiation window up to 405 nm, or electron beam sources. The arrangement of these sources is known in principle and can be adapted to the circumstances of the workpiece and the process parameters. In the case of workpieces of complex shape, such as are envisaged for automobile bodies, areas not accessible to direct radiation (shadow regions) such as cavities, folds and other structural undercuts can be (partly) cured using pointwise, small-area or all-round emitters in conjunction with an automatic movement means for the irradiation of cavities or edges.

The equipment and conditions for these curing methods are described, for example, in R. Holmes, U. V. and E. B. Curing Formulations for Printing Inks, Coatings and Paints, SITA Technology, Academic Press, London, United Kingdom 1984.

Curing here may take place in stages, i.e., by multiple exposure to light or actinic radiation. It may also take place alternately, i.e., by curing alternately with UV radiation and electron beams.

Where thermal curing and actinic radiation curing are employed together (dual cure), these methods may be employed simultaneously or alternately. Where the two curing methods are used alternately, it is possible, for example, to begin with the thermal cure and to end with the actinic radiation cure. In other cases it may prove advantageous to begin and to end with the actinic radiation cure.

The process of the invention provides multicoat paint systems which are superior in surface smoothness, corrosion protection effect, substrate adhesion, intercoat adhesion, stonechip resistance, edge protection effect, weathering stability, and chemical resistance to multicoat paint systems which have not been produced by an inventive procedure.

The multicoat paint systems of the invention therefore have a particularly high quality and a long service life, even under extreme climatic conditions, so making them especially attractive both economically and technically to users.

### INVENTIVE AND COMPARATIVE EXAMPLES

### Preparation Example 1

### Preparation of a Crosslinking Agent for an Electrocoat Material

A reactor was charged with 10 462 parts of isomers and higher-functional oligomers based on 4,4′-diphenylmethane diisocyanate, having an NCO equivalent weight of 135 g/eq (Lupranat^{R} M20S from BASF AG; NCO functionality about 2.7; 2,2′- and 2,4′-diphenylmethane diisocyanate content less than 5%) under a nitrogen atmosphere. 20 parts of dibutyltin dilaurate were added and 9 626 parts of butyl diglycol were added dropwise at a rate such that the product temperature remained below 60° C. After the end of the addition, the temperature was held at 60° C. for a further 60 minutes and an NCO equivalent weight of 1 120 g/eq was measured (based on solid fractions). Following dilution in 7 737 parts of methyl isobutyl ketone and addition of 24 parts of dibutyltin dilaurate, 867 parts of melted trimethylolpropane were added at a rate such that a product temperature of 100° C. was not exceeded. After the end of the addition, the action was allowed to continue for 60 minutes. The product was cooled to 65° C. and diluted simultaneously with 963 parts of n-butanol and 300 parts of methyl isobutyl ketone. The solids content was 70.1% (1 hour at 130° C.).

### Preparation Example 2

### Preparation of a Precursor of the Amine Component for an Electrocoat Binder

The water of reaction was removed at from 110 to 140° C. from a 70% strength solution of diethylenetriamine in methyl isobutyl ketone. The solution was then diluted with methyl isobutyl ketone until it had an amine equivalent weight of 131 g/eq.

### Preparation Example 3

### Preparation of the Aqueous Electrocoat Binder Dispersion D1

In a reactor fitted with stirrer, reflux condenser, internal thermometer, and inert gas inlet, 6 150 parts of epoxy resin based on bisphenol A having an epoxy equivalent weight (EEW) of 188 g/eq, together with 1 400 parts of bisphenol A, 355 parts of dodecylphenol, 470 parts of p-cresol and 441 parts of xylene, were heated to 125° C. under a nitrogen atmosphere and stirred for 10 minutes. The mixture was subsequently heated to 130° C. and 23 parts of N,N-dimethylbenzylamine were added. The reaction mixture was held at this temperature until the EEW had reached a level of 880 g/eq.

At that point a mixture of 7 097 parts of a crosslinking agent from Preparation Example 1 and 90 parts of the additive K 2000 (polyether from Byk Chemie) were added and the resulting mixture was held at 100° C. Half an hour after adding the crosslinker, 211 parts of butyl glycol and 1 210 parts of isobutanol were added. Immediately thereafter a mixture of 467 parts of the precursor from Preparation Example 2 and 520 parts of methyl ethanol amine were placed in the reactor and the batch was conditioned at a temperature of 100° C. After a further half an hour, the temperature was raised to 105° C. and 150 parts of N,N-dimethylaminopropylamine were added.

75 minutes following addition of the amine, 903 parts of Plastilit® 3060 (propylene glycol compound from BASF Aktiengesellschaft) were added, and the mixture was diluted with 522 parts of propylene glycol phenyl ether (mixture of 1-phenoxy-2-propanol and 2-phenoxy-1-propanol from BASF Aktiengesellschaft) and cooled to 95° C.

After 10 minutes, 14 821 parts of the reaction mixture were transferred to a dispersing vessel. In that vessel 474 parts of lactic acid (88% strength in water), in solution in 7 061 parts of water, were added. The batch was subsequently homogenized for 20 minutes before being diluted further with an additional 12 600 parts of water in small portions.

By vacuum distillation the volatile solvents were removed and then replaced in equal quantity by water.

The dispersion D1 had the following characteristics:

| | |
|---|---|
| Solids content: | 33.8% by weight (1 hour at 130° C.) |
| | 29.9% by weight (0.5 hour at 180° C.) |
| Base content: | 0.71 milliequivalents/g solids |
| Acid content: | 0.36 milliequivalents/g solids |
| pH: 6.3 | |
| Particle size: | 113 nm (determined by photon correlation |
| | spectroscopy) |

### Preparation Example 4

### Preparation of the Aqueous Electrocoat Binder Dispersion D2

To prepare the binder dispersion D2, Preparation Example 3 was repeated except that immediately following dilution with propylene glycol phenyl ether 378 parts of bismuth 2-ethylhexanoate (K-KAT 348 from King Industries) were admixed with stirring. After cooling, 14 821 parts of the reaction mixture were dispersed in water in analogy to Preparation Example 3.

The dispersion D2 had the following characteristics:

| | |
|---|---|
| Solids content: | 33.9% by weight (1 hour at 130° C.) |
| | 30.1% by weight (0.5 hour at 180° C.) |
| Base content: | 0.74 milliequivalents/g solids |
| Acid content: | 0.48 milliequivalents/g solids |
| pH: 5.9 | |
| Particle size: | 189 nm (determined by photon correlation |
| | spectroscopy) |

### Preparation Example 5

### Preparation of an Epoxy-amine Adduct Solution

In accordance with European patent EP 0 505 445 B1, Example 1.3, an organic-aqueous solution of epoxy-amine adduct was prepared by reacting in a first stage 2 598 parts of bisphenol A diglycidyl ether (EEW: 188 g/eq), 787 parts of bisphenol A, 603 parts of dodecylphenol and 206 parts of butyl glycol in the presence of 4 parts of triphenylphosphine at 130° C. to an EEW of 865 g/eq. While it cooled, the reaction mixture was diluted with 849 parts of butyl glycol and 1 534 parts of D.E.R. 732 (polypropylene glycol diglycidyl ether from DOW Chemical) and at 90° C. it was left to react further with 266 parts of 2-(2′-aminoethoxy)ethanol and 212 parts of N,N-dimethylaminopropylamine. After 2 hours the viscosity of the resin solution was constant [5.3 dpa·s; 40% strength in Solvenon® PM (methoxypropanol from BASF Aktiengesellschaft); cone and plate viscometer at 23° C.]. It was diluted with 1 512 parts of butyl glycol and the base groups were partly neutralized with 201 parts of glacial acetic acid, followed by further dilution with 1 228 parts of deionized water, after which the solution was discharged. This gave a 60% strength aqueous-organic resin solution whose 10% dilution had a pH of 6.0.

The epoxy-amine adduct solution was subsequently used to prepare a dispersion for inventive use.

### Preparation Example 6

### The Preparation of a Dispersion D3 for Inventive Use

As described in international patent application WO 98/07794, Example 4.3, a stainless steel reaction vessel was charged with 18 873 parts of the epoxy-amine adduct solution from Preparation Example 5 and 37 532 parts of deionized water and 5 000 parts of ethanol. The reactor contents were heated to 80° C. and 383 parts of tert-butyl per-2-ethylhexanoate initiator were added. Over the course of one hour 38 411 parts of styrene were added. The temperature was held at 90° C. Four hours after the addition of styrene, the reaction had ended.

The dispersion D3 had the following characteristics:

| | |
|---|---|
| Solids content: | 50% by weight (1 hour at 130° C.) |
| Viscosity: | 280 mPa.s |

### Preparation Example 7

### The preparation of a Pigment Paste P1

First of all, a premix was formed from 277 parts of water and 250 parts of the epoxy-amine adduct solution described in Preparation Example 5. The 5 parts of carbon black, 67 parts of Extender ASP 200, 373 parts of titanium dioxide (TI-PURE R900 from DuPont) and 25 parts of crosslinking catalyst (DBTO: dibutyltin oxide) were added. The resulting mixture was homogenized for 30 minutes using a high-speed dissolver-stirrer mechanism. The mixture was subsequently dispersed in a stirred laboratory mill for from 1 to 1.5 hours to a Hegman fineness of 12 and was adjusted with further water to the desired processing viscosity.

### Examples 1 to 4 (Inventive) and C1 and C2 (Comparative)

### Preparation of Cathodically Depositable Electrocoat Materials for Inventive Use (Examples 1 to 4) and Noninventive Use (Examples C1 and C2)

The electrocoat materials compiled in Table 1 were prepared from the electrocoat binder dispersions D1 and D2 from Preparation Examples 3 and 4, the dispersion D3 for inventive use from Preparation Example 6 and the pigment paste P1 from Preparation Example 7. In the case of the pigmented electrocoat materials of Example 1 and Example C1, the solids content is approximately 20% by weight. In the case of the unpigmented electrocoat materials of Examples 1 to 4 and of Example C2, the solids content is approximately 15% by weight.

### Table 1

### The Composition in Parts by Weight of the Electrocoat Materials for Inventive Use (Examples 1 to 4) and for Noninventive Use (Examples C1 and C2)

| | Examples: | | | | | |
|---|---|---|---|---|---|---|
| Constituents | 1 | 2 | 3 | 4 | C1 | C2 |
| D1 | 2 332 | — | — | — | 2 771 | — |
| D2 | — | 2 118 | 1 994 | 1 869 | — | 2 492 |
| D3 | 257 | 225 | 300 | 375 | — | — |
| P1 | 313 | — | — | — | 313 | — |
| Water | 2 098 | 2 657 | 2 706 | 2 756 | 1 916 | 2 098 |

### Examples 5 to 8 (Inventive) and C4 to C11 (Comparative)

### Production of Multicoat Paint Systems Inventively (Examples 5 to 8) and Noninventively (Examples C4 to C11)

Compilation 1 gives an overview of which electrocoat materials were employed for Examples 5 to 8 and for Examples C4 to C11.

Compilation 1

| Inventive | Comparative | | |
|---|---|---|---|
| Example | Example | Electrocoat material | |
| 5 | C6 | 1 | — |
| 6 | C9 | 2 | — |
| 7 | C10 | 3 | — |
| 8 | C11 | 4 | — |
| — | C4, C5 | — | C1 |
| — | C7, C8 | — | C2 |

After bath aging of 24 hours, the electrocoat materials were deposited on steel panels (BO 26 W 42 OC from Chemetall) which had not been given a passivating rinse. The deposition voltage and deposition temperature (300 to 330 volts, bath temperature 30° C.) were chosen so that baking for 15 minutes at a panel temperature of 185° C. resulted in electrocoats having a thickness of from 17 to 20 μm.

In one test series A (i.e., Examples C4, C6, C7, C9, C10, and C11), the electrocoats were baked following their application under the stated conditions. Thereafter the electrocoats were overcoated with an OEM aqueous surfacer (FU80-7211 from BASF Coatings AG). The surfacer film was baked at 155° C. under conventional conditions, to give surfacer coats with a thickness of from 30 to 35 μm. The surfacer coats were further coated by the wet-on-wet technique with a commercially customary aqueous basecoat material from BASF Coatings AG and with a commercially customary clearcoat material from BASF Coatings AG, so that baking resulted in aqueous basecoats with a thickness of from 20 to 25 μm and clearcoats with a thickness of from 40 to 50 μm.

In a test series B (i.e., Examples 5, 6, 7, 8, C5 and C8) the electrocoats were not baked but instead predried at 90° C. for 10 minutes. The abovementioned OEM aqueous surfacer FU80-7211 was applied to the dried electrocoats, after which the two films were baked for 15 minutes at a panel temperature of 185° C. After that, as described above, overcoating was carried out by the wet-on-wet technique with an aqueous basecoat and a clearcoat.

The resultant multicoat paint systems of test series A and B were subjected to the VDA stonechip test, which is known in the art, and to the MB ball impact test, which is known in the art. The results can be found in Table 2.

The results of the tests show that in the case of the multicoat paint systems whose electrocoat materials did not contain dispersion D3 for inventive use the stonechip resistance and corrosion protection effect was substantially independent of the nature of their production (cf. Examples C4 and C5 and also C7 and C8). By contrast, in the case of the multicoat paint systems whose electrocoat materials contained the dispersion D3 for inventive use, the stonechip resistance and corrosion protection effect tended to be even better when using the process of the invention [cf. Example 5 with Example C6 (pigmented electrocoats) and Example 6 and Example C9, Example 7 and Example C10, Example 8 and Example C11 (unpigmented electrocoats)].

TABLE 2
Stonechip resistance and corrosion protection effect of
multicoat paint systems produced inventively
(Examples 5 to 8) and noninventively
(Examples C4 to C11)

| | Examples: | | | | | | | | | | | |
|---|---|---|---|---|---|---|---|---|---|---|---|---|
| | 5 | 6 | 7 | 8 | C4 | C5 | C6 | C7 | C8 | C9 | C10 | C11 |
| Series | B | B | B | B | A | B | A | A | B | A | A | A |
| VDA | 2 | 2 | 2.5 | 3 | 2 | 2 | 2 | 2 | 2 | 2 | 2 | 2 |
| MB | | | | | | | | | | | | |
| Flaking | 6 | 4 | 6 | 7 | 7 | 9 | 8 | 7 | 10 | 7 | 8 | 6 |
| Rusting | 1 | 4 | 1 | 1 | 5 | 5 | 3 | 5 | 5 | 2 | 2 | 1 |

### Examples 9 to 12 (Inventive) and C12 and C13 (Comparative)

### Production of Multicoat Paint Systems Inventively (Examples 9 to 12) and Noninventively (Examples C12 and C13)

Compilation 2 gives an overview of which electrocoat materials were employed for Examples 9 to 12 and for Examples C12 and C13.

Compilation 2

| Inventive | Comparative | | |
|---|---|---|---|
| Example | Example | Electrocoat material | |
| 9 | — | 1 | — |
| 10 | — | 2 | — |
| 11 | — | 3 | — |
| 12 | — | 4 | — |
| — | C12 | — | C1 |
| — | C13 | — | C2 |

After bath aging of 24 hours, the above-described electrocoat materials were deposited on steel panels (BO 26 W 42 OC from Chemetall) which had not been given a passivating rinse. The deposition voltage and deposition temperature (300 to 330 volts, bath temperature 30° C.) were chosen so that baking for 15 minutes at a panel temperature of 185° C. resulted in electrocoats having a thickness of from 17 to 20 μm.

In test series C, the electrocoat films were overcoated, in one case by the wet-on-wet technique and in the other case after baking (standard technique), with the commercially customary aqueous OEM surfacer (FU80-7211 from BASF Coatings AG). In all cases, surfacer coats with a thickness of from 30 to 35 μm were obtained. Surface profile measurements (Pa in μm) were carried out using a Perthometer S8P from Mahr. Comparison between the effect of the wet-on-wet technique for inventive use and of the standard technique on the surface smoothness was drawn on the basis of the differences between the Pa figures for the standard technique and those for the wet-on-wet technique. This rules out the fluctuation of the individual figures owing to overcoating effects, since each electrocoat was overcoated separately.

The results can be found in Table 3.

In a test series D, the electrocoat films were overcoated in one case by the wet-on-wet technique and in the other case after baking (standard technique) with an aqueous surfacer (Ecoprime® FU 30-7210 from BASF Coatings AG). Thereafter a commercially customary aqueous basecoat material (Ecostar®) and a commercially customary clearcoat material (both from BASF Coatings AG) were applied by the wet-on-wet technique and baked.

This gave multicoat paint systems of the following structure:

electrocoat from 17 to 20 μm

functional coat from 12 to 17 μm

basecoat from 20 to 25 μm

clearcoat from 40 to 50 μm.

The surface smoothness was measured and evaluated as described above. The results can likewise be found in Table 3.

The results compiled in Table 3 demonstrate the following:

1. Wet-on-wet techniques lead fundamentally to a better surface smoothness than the standard technique.

2. Electrocoat materials which contain the inventive dispersions D3 give smoother surfaces both in the standard technique and in the wet-on-wet technique than electrocoat materials without dispersion D3.

3. Electrocoat materials containing inventive dispersions D3 and processed by the wet-on-wet technique give the smoothest surfaces.

TABLE 3
The surface smoothness of the multicoat paint systems of
Examples 9 to 10 and Examples C12 and C13

| | Examples | | | | | |
|---|---|---|---|---|---|---|
| | 9 | 10 | 11 | 12 | C12 | C13 |
| Series: | C/D | C/D | C/D | C/D | C/D | C/D |
| Pa difference: | 0.28/0.14 | 0.13/0.05 | 0.08/0.04 | 0.07/0.01 | 0.25/0.1 | 0.12/0.01 |

What is claimed is:

1. A process for producing multicoat paint systems on electroconductive substrates by a wet-on-wet technique, in which
(I) a cathodically depositable electrocoat material is deposited on the electroconductive substrate,
(II) at least one coating material curable thermally or both thermally and with actinic radiation is applied to the resultant electrocoat film, and then
(III) the electrocoat film and the film of the coating material, or the two said films and at least one further, overlying film of a coating material, are jointly cured,
wherein the cathodically depositable electrocoat material comprises an aqueous dispersion preparable by
(1) polymerizing an ethylenically unsaturated monomer or a mixture of ethylenically unsaturated monomers in
(2) an aqueous solution of an at least partly protonated epoxy-amine adduct,
(3) the epoxy-amine adduct being obtainable by reacting
(A) at least one glycidyl ether of a polyphenol, containing on average at least one epoxide group in the molecule,
(B) at least one polyglycidyl ether of a polyol, containing on average more than 1.0 epoxide group in the molecule, and
(C) at least one compound containing a primary amino group in the molecule,
to give the epoxy-amine adduct, components (A) and (B) being used in an equivalents ratio of from 1.0:0.5 to 1.0:8.0, and using from 0.3 to 0.7 mol of component (C) per equivalent of epoxide groups of (A) and (B).

2. The process as claimed in claim 1, wherein the aqueous dispersion is obtainable by using the ethylenically unsaturated monomer or mixture of ethylenically unsaturated monomers and the at least partly protonated epoxy-amine adduct in a weight ratio of from 9.0:1.0 to 0.1:1.0.

3. The process as claimed in claim 1 or 2, wherein the aqueous dispersion is obtainable using styrene as the ethylenically unsaturated monomer.

4. The process as claimed in claim 1 or 2, wherein the aqueous dispersion is obtainable using a mixture of styrene and at least further unsaturated monomer copolymerizable with styrene, as the mixture of ethylenically unsaturated monomers.

5. The process as claimed in claim 4, wherein the mixture of ethylenically unsaturated monomers contains at least 70% by weight styrene.

6. The process as claimed in any of claims 1 to 5, wherein the coating material is a pigmented or unpigmented coating material.

7. The process as claimed in any of claims 1 to 6, wherein the coating material is self-crosslinking.

8. The process as claimed in any of claims 1 to 7, wherein the coating material is externally crosslinking.

9. The process as claimed in claim 8, wherein the coating material is a one-component system or a two-component or multicomponent system.

10. The process as claimed in any of claims 1 to 9, wherein the coating material is substantially free from water and/or organic solvents and is pulverulent or liquid (100% system).

11. The process as claimed in any of claims 1 to 9, wherein the coating material is a water-based coating material.

12. The process as claimed in claim 11, wherein the coating material is an aqueous powder coating dispersion.

13. The process as claimed in any of claims 1 to 9, wherein the coating material is a conventional coating material, i.e., a coating material based on organic solvents.

14. The process as claimed in any of claims 1 to 13, wherein prior to the application of the coating material the electrocoat film is dried without being crosslinked.

15. The process as claimed in any of claims 1 to 14, wherein the electroconductive substrates are bodies of motor vehicles or parts thereof, furniture and industrial components, including coils and containers.
